(19) United States
(12) Patent Application Publication
(10) Pub. No.: US 2015/0041675 A1
(43) Pub. Date: Feb. 12, 2015

(54) AIR BLOWER, ION TRANSMITTING DEVICE, ELECTRICAL APPLIANCE, AND REMOTE CONTROL HOLDING STRUCTURE

(71) Applicant: Sharp Kabushiki Kaisha, Osaka-shi, Osaka (JP)

(72) Inventors: Masaki Shibata, Osaka-shi (JP); Kenichi Shiraishi, Osaka-shi (JP); Nobuhiro Iwaki, Osaka-shi (JP)

(21) Appl. No.: 14/386,051

(22) PCT Filed: Apr. 2, 2013
(86) PCT No.: PCT/JP2013/060064

§ 371 Date: Sep. 18, 2014

(30) Foreign Application Priority Data
Apr. 3, 2012 (JP) 2012-084749
Apr. 3, 2012 (JP) 2012-084758
Apr. 3, 2012 (JP) 2012-084762

(51) Int. Cl. A61L 9/22; F04D 29/42; F04D 29/62; H01J 27/02

(52) U.S. Cl. CPC A61L 9/22; H01J 27/022; F04D 29/4226; F04D 29/624
USPC 250/423 R; 415/211.2; 415/121.3

(57) ABSTRACT

An inlet port 4 and an outlet port 5 which are open in a body housing 2, an air-blowing pathway 20 disposed inside the body housing 2, making the inlet port 4 and the outlet port 5 communicate with each other, and also having a plurality of divisional pathways 22 obtained by division with partition plates 21, an air-blowing fan 7 disposed upstream from the divisional pathways 22 inside the air-blowing pathway 20, a removable inner cover 10 covering the divisional pathways 22 and having an inner surface which forms a side wall of the air-blowing pathway 20, and a removable exterior cover 9 covering the inner cover 10 to form an outer surface of the body housing 2 are provided, and the inner cover 10 has marking portions 11 formed on an outer surface along the partition plates 21.

### TECHNICAL FIELD

The present invention relates to an air blower which transmits an airflow. The present invention also relates to an ion transmitting device which transmits an airflow containing ions. Furthermore, the present invention relates to a structure which holds a remote control for performing a remote operation and an electrical appliance with the structure.

### BACKGROUND ART

A conventional air blower is disclosed in PTL 1. This air blower includes a body housing set on a floor surface, and an inlet port and an outlet port are open in the body housing. Inside the body housing, an air-blowing duct is formed which makes the inlet port and the outlet port communicate with each other, and an air-blowing fan is disposed inside the air-blowing duct. A removable filter is disposed at the inlet port, and a plurality of airflow direction plates which change the airflow direction are provided at the outlet port.

In the above-structured air blower, indoor air flows from the inlet port into the inside of the air-blowing duct by driving the air-blowing fan. The air distributed through the air-blowing duct is transmitted from the outlet port in a direction corresponding to the orientation of the airflow direction plates. With this, air is blown toward a user, and indoor air can be circulated.

Also, a conventional ion transmitting device is disclosed in PTL 2. This ion transmitting device includes an ion generating unit provided with two ion generators (ion generating portions). The ion generating unit can be attached to and removed from a unit inserting portion that is open in a casing, which is a body housing.

Furthermore, conventional remote control holding structures are disclosed in PTL 3 and PTL 4. A remote control holding structure described in PTL 3 includes a remote control holder fixed to an indoor wall surface. The remote control holder has a recessed portion which can accommodate a lower part of a remote control, and the remote control can be removably accommodated in this recessed portion. With this, a loss of the remote control can be prevented.

Also, a remote control holding structure described in PTL 4 includes a remote control holder fixed to an indoor wall surface. Recessed engaging portions are provided at two positions on the rear surface of a remote control, and projecting engagement receiving portions are provided at two positions on the front surface of the remote control holder. With the engaging portions and the engagement receiving portions engaged together, the remote control is held. With this, a loss of the remote control can be prevented, and falling of the remote control can be prevented even when the remote control is pushed downward or diagonally upward.

### CITATION LIST

### Patent Literature

PTL 1: Japanese Unexamined Patent Application Publication No. 2-185696

PTL 2: Japanese Unexamined Patent Application Publication No. 2012-21683

PTL 3: Japanese Unexamined Patent Application Publication No. 7-158945

PTL 4: Japanese Unexamined Patent Application Publication No. 2008-133586

### SUMMARY OF INVENTION

### Technical Problem

According to the air blower of PTL 1 described above, fine dust particles passing through the filter disposed at the inlet port are disadvantageously deposited inside an air-blowing pathway to decrease air-blowing efficiency.

Moreover, according to the ion transmitting device of PTL 2 described above, when the ion generator is replaced, the ion generator has to be removed from the ion generating unit after the ion generating unit is removed from the casing. With this, disadvantageously, it is troublesome to replace the ion generator, and there is a possibility of losing the ion generating unit.

Furthermore, according to the remote control holding structures of PTL 3 and PTL 4 described above, a remote control holder is required separately from an electrical appliance body, and therefore the number of components disadvantageously increases.

An object of the present invention is to provide an air blower capable of preventing a decrease in air-blowing efficiency. Another object of the present invention is to provide an ion transmitting device capable of easily replacing an ion generator and preventing a loss of the ion generator. Still another object of the present invention is to provide a remote control holding structure capable of reducing the number of components and an electrical appliance with the structure.

### Solution to Problem

To achieve the object described above, an air blower of the present invention includes a body housing having an inlet port and an outlet port which are open, an air-blowing duct disposed inside the body housing, making the inlet port and the outlet port communicate with each other, and also having a plurality of divisional pathways obtained by division with partition plates, an air-blowing fan disposed upstream from the divisional pathways inside the air-blowing duct, a removable inner cover covering the divisional pathways and having an inner surface which forms a side wall of the air-blowing duct, and a removable exterior cover covering the inner cover to form an outer surface of the body housing, wherein the inner cover has marking portions formed on an outer surface along the partition plates.

According to this structure, when the air-blowing fan is driven, indoor air flows from the inlet port into the air-blowing pathway. The air flowing in the air-blowing pathway is distributed into the plurality of divisional pathways obtained by division with the partition plates downstream of the air-blowing fan, and is transmitted from the outlet port. When the exterior cover and the inner cover are removed, the air-blowing pathway including the divisional pathways can be cleaned. The marking portions formed along the partition plates are provided on the outer surface of the inner cover, and the inner cover is mounted so that the marking portions are superposed on the partition plates.

Also, in the above-structured air blower of the present invention, the inlet port is provided in a lower part of the body housing and the outlet port is provided in a front surface of the body housing above the inlet port, and the partition plates have a lower part extending in a vertical direction and an upper part curved toward the outlet port.

According to this structure, air flows in the air-blowing pathway from the inlet port disposed on the lower part of the body housing. The air flowing in the air-blowing pathway is distributed upward along the partition plates, and is curved forward to be transmitted from the outlet port.

Furthermore, in the above-structured air blower of the present invention, fitting grooves fitting in the partition plates are provided in the inner surface of the inner cover, and rear surfaces of the fitting grooves project from an outer surface of the inner cover to form the marking portions. According to this structure, with the partition plates fitting in the fitting grooves, the inner cover is mounted. The rear surface of the fitting groove projects from the outer surface of the inner cover to form the marking portion along the partition plate.

Still further, in the above-structured air blower of the present invention, the marking portions each have a rib obtained by extending a side wall of the fitting groove and projecting from the rear surface of the fitting groove.

Still further, in the above-structured air blower of the present invention, the exterior cover is formed on a curved surface projecting outward and has reinforcing ribs projecting from an inner surface and extending in a direction crossing the partition plates, and the reinforcing ribs abut on the marking portions when the exterior cover is mounted.

According to this structure, the exterior cover forming the outer surface of the body housing is curved, and the reinforcing rib provided on the inner surface of the exterior cover abuts on the marking portion projecting from the outer surface of the inner cover. With this, deformation of the exterior cover is prevented.

Still further, in the above-structured air blower of the present invention, a downstream flow-path area of the divisional pathways is narrowed by the inner cover with respect to an upstream, and the marking portion facing an upstream part of the divisional pathways has a groove portion provided by extending both side walls of the fitting groove and formed on the outer surface of the inner cover.

According to this structure, the flow-path area of the divisional pathways on the downstream side is narrowed by the inner cover, and the air velocity of the airflow transmitted from the outlet port is increased. In the outer surface of the inner cover facing the upstream portion of the divisional pathways, groove portions formed by extending both side walls of the fitting groove are provided as being recessed. With the groove portions, the marking portions along the partition plates are formed.

Also, a remote control holding structure of the present invention is a remote control holding structure which accommodates and holds a remote control for performing a remote operation, the structure including a single insertion port which is open in an exterior of a body housing and allows the remote control and a human finger to be simultaneously inserted therein and a holding hole extending from the insertion port toward inside of the body housing, wherein the holding hole has a remote control holding portion in a lower part and a hand-hook portion in an upper part, the remote control holding portion extending from the insertion port in a diagonally downward direction to hold the remote control and the hand-hook portion extending from the insertion port in a lateral direction or a diagonally upward direction and in which the human finger is hooked when the body housing is carried.

According to this structure, the holding hole is formed by integrating the remote control holding portion which holds the remote control and the hand-hook portion in which a human finger is hooked at the time of carrying the electrical appliance. With this, the number of components regarding the remote control holding portion and the hand-hook portion is reduced, and occupying space is also decreased.

Also, in the above-structured remote control holding structure of the present invention, a projecting portion projecting toward the insertion port side is provided inside of the holding hole, and sectioning into the remote control holding portion and the hand-hook portion is made by the projecting portion.

Furthermore, in the above-structured remote control holding structure of the present invention, the holding hole is formed so that a vertical cross-section shape along a direction of inserting the remote control is a trapezoidal shape.

Still further, in the above-structured remote control holding structure of the present invention, the holding hole is formed to have a size allowing the remote control accommodated in the holding hole to partially project outward from the insertion port.

Also, an ion transmitting device of the present invention includes a body housing having an inlet port and outlet port which are open, an air-blowing duct making the inlet port and the outlet port communicate with each other, an air-blowing fan which distributes an airflow into the air-blowing duct, an ion generator having an ion generating portion which transmits ions to the air flow distributed through the air-blowing duct, a window portion formed of an opening provided on a side plate forming one surface of the air-blowing duct, and a holder which removably holds the ion generator, wherein the holder can rotate so as to take a position where the ion generating portion faces inside of the air-blowing duct via the window portion and a position where the ion generating portion is separated away from the window portion.

According to this structure, the holder which removably holds the ion generator rotates so as to take a position where the ion generating portion faces the inside of the air-blowing duct via the window portion and a position where the ion generating portion is separated away from the window portion. With this, at the time of replacing the ion generator, the holder is not removed from the body housing of the ion transmitting device.

Furthermore, in the above-structured ion transmitting device of the present invention, the air-blowing fan is formed of a centrifugal fan, the air-blowing duct is led from the air-blowing fan to have an imbalance in one decentering direction with respect to a rotation axis line of the air-blowing fan, and a holder coupling portion which rotatably supports the holder is disposed outside a direction opposite to the decentering direction with respect to the air-blowing duct.

According to this structure, the exhaust port of the air-blowing fan formed of a centrifugal fan is disposed so as to have an imbalance to, for example, the rear part of the body housing, with respect to the rotation axis line, and the air-blowing duct is led from the air-blowing fan to have an imbalance rearward. The holder coupling portion is disposed forward and outside of the air-blowing duct.

Still further, in the above-structured ion transmitting device of the present invention, the holder includes a guide portion which causes the ion generator to make a sliding movement when the ion generator is attached or removed.

Still further, in the above-structured ion transmitting device of the present invention, the guide portion causes the ion generator to make a sliding movement in a direction crossing a center axis line of rotation of the holder.

Still further, in the above-structured ion transmitting device of the present invention, the holder includes a connector portion which is electrically connected to the ion generator together with insertion of the ion generator into the holder. According to this structure, when mounted on the holder, the ion generator is electrically connected via the connector portion.

Still further, in the above-structured ion transmitting device of the present invention, the ion generator includes a plurality of said ion generating portions, and the plurality of said ion generating portions are aligned in a direction crossing an airflow distributing direction of the air-blowing duct.

Still further, an ion transmitting device of the present invention includes the above-structured air blower, wherein an ion generator is provided which transmits ions toward the air-blowing duct on a downstream side of the air-blowing fan. According to this structure, ions are transmitted inside the air-blowing pathway by driving the ion generator, and an airflow containing ions is transmitted from the outlet port.

Still further, an ion transmitting device of the present invention includes the above-structured remote control holding structure, a remote control holding structure, and an ion generator which generates ions, wherein the ion transmitting device transmits an airflow containing ions.

Still further, in the above-structured ion transmitting device of the present invention, an air-blowing duct is provided which makes an inlet port which is open in a lower part of the body housing and an outlet port which is open in an upper front surface communicate with each other, the air-blowing duct extends upward from the inlet port and is curved forward to be coupled to the outlet port, and the remote control holding structure is provided to an upper rear surface of the body housing. According to this structure, the remote control holding structure is formed in a vacant space behind the air-blowing duct curved forward from the inlet port of the lower part of the body housing toward the outlet port of the upper front surface.

Also, an electrical appliance of the present invention includes the above-structured remote control holding structure.

### Advantageous Effects of Invention

According to the present invention, the air-blowing pathway having the divisional pathways obtained by division with the partition plates is provided inside the body housing with its outer surface formed of the removable exterior cover. Also, the removable inner cover covering the divisional pathways has the marking portions along the partition plates. With this, the inside of the air-blowing pathway including the divisional pathways can be cleaned by removing the exterior cover and the inner cover. Therefore, a decrease in air-blowing efficiency of the ion transmitting device can be prevented, and the transmitted airflow can be also kept clean. Furthermore, after cleaning of the air-blowing pathway, the inner cover can be easily mounted by superposing the marking portions on the partition plates, and usability of the air blower can be improved.

Also, according to the present invention, the holder which holds the ion generator rotates so as to take a position where the ion generating portion faces the inside of the air-blowing duct via the window portion and a position where the ion generating portion is separated away from the window portion. With this, an ion transmitting device capable of easily replacing the ion generator and preventing a loss of the holder of the ion generator can be provided.

Furthermore, according to the present invention, the remote control holding structure is formed of the holding hole having the remote control holding portion and the hand-hook portion. With this, the number of components of the remote control holding structure can be reduced, and occupying space can be decreased. Therefore, the size and cost of the electrical appliance with the remote control holding structure can be reduced.

### BRIEF DESCRIPTION OF DRAWINGS

FIG. 1 is a perspective view of an ion transmitting device of a first embodiment of the present invention when viewed from the front.

FIG. 2 is a side view of the ion transmitting device of the first embodiment of the present invention with an exterior cover and a side plate removed therefrom.

FIG. 3 is a partially-enlarged side view of the ion transmitting device of the first embodiment of the present invention with the exterior cover and the side plate removed therefrom.

FIG. 4 is a top view of an ion generator of the ion transmitting device of the first embodiment of the present invention.

FIG. 5 is a side view of the ion generator of the ion transmitting device of the first embodiment of the present invention.

FIG. 6 is a perspective view of the ion generator of the ion transmitting device of the first embodiment of the present invention.

FIG. 7 is a top view of the ion transmitting device of the first embodiment of the present invention.

FIG. 8 is a perspective view of the ion transmitting device of the first embodiment of the present invention with the exterior cover removed therefrom.

FIG. 9 is a front view of a holder of the ion transmitting device of the first embodiment of the present invention.

FIG. 10 is a perspective view of the holder of the ion transmitting device of the first embodiment of the present invention.

FIG. 11 is a front view of the ion generator and the holder of the ion transmitting device of the first embodiment of the present invention.

FIG. 12 is a rear view of the ion generator and the holder of the ion transmitting device of the first embodiment of the present invention.

FIG. 13 is a front view depicting an inner cover of the ion transmitting device of the first embodiment of the present invention.

FIG. 14 is an A-A sectional view of FIG. 13.

FIG. 15 is a B-B sectional view of FIG. 13.

FIG. 16 is a partially-enlarged side view of the ion transmitting device of the first embodiment of the present invention with the exterior cover removed therefrom.

FIG. 17 is a partially-enlarged side view of an ion transmitting device of a second embodiment of the present invention with an exterior cover removed therefrom.

### DESCRIPTION OF EMBODIMENTS

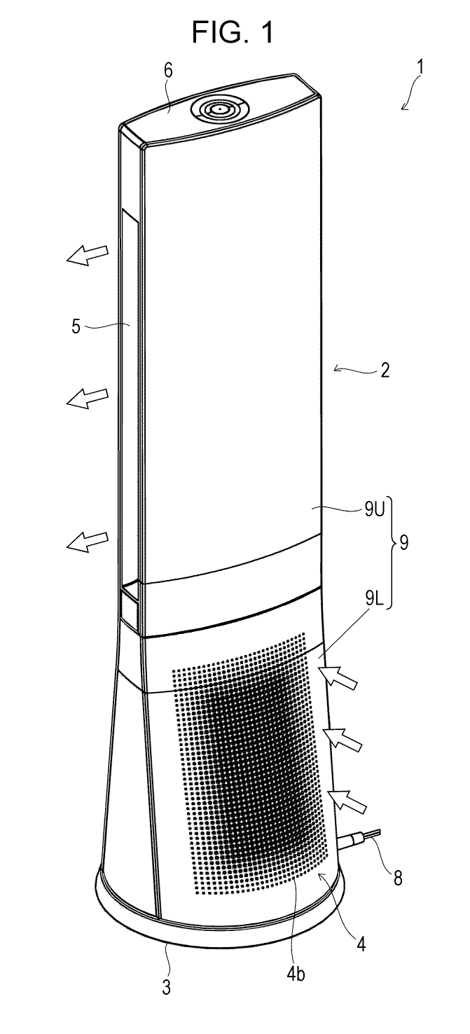

Embodiments of the present invention are described below with reference to the drawings. FIG. 1 is an external perspective view of an ion transmitting device of a first embodiment. In the drawing, hollow arrows indicate an air distributing direction. An ion transmitting device 1 includes a body housing 2 in a columnar shape and a mount portion 3 disposed below the body housing 2.

The body housing 2 is formed in a columnar shape extending vertically and longitudinally. The body housing 2 has a circular bottom surface, and has a part from a substantially center part in a vertical direction to an upper end formed substantially in a rectangular parallelepiped shape. The upper part substantially in a rectangular parallelepiped shape has a front-to-rear length longer than a breadth, and both left and right side surfaces are curved to swell outward.

Both side surfaces of the upper part of the body housing 2 are each covered with an upper exterior cover 9U, which is a removable resin-molded article with a curved surface projecting outward. One side surface of the lower part of the body housing 2 is covered with a lower exterior cover 9L, which is a removable resin-molded article with an inlet port 4 open. In the following description, the upper exterior cover 9U and the lower exterior cover 9L may be collectively referred to as an exterior cover 9.

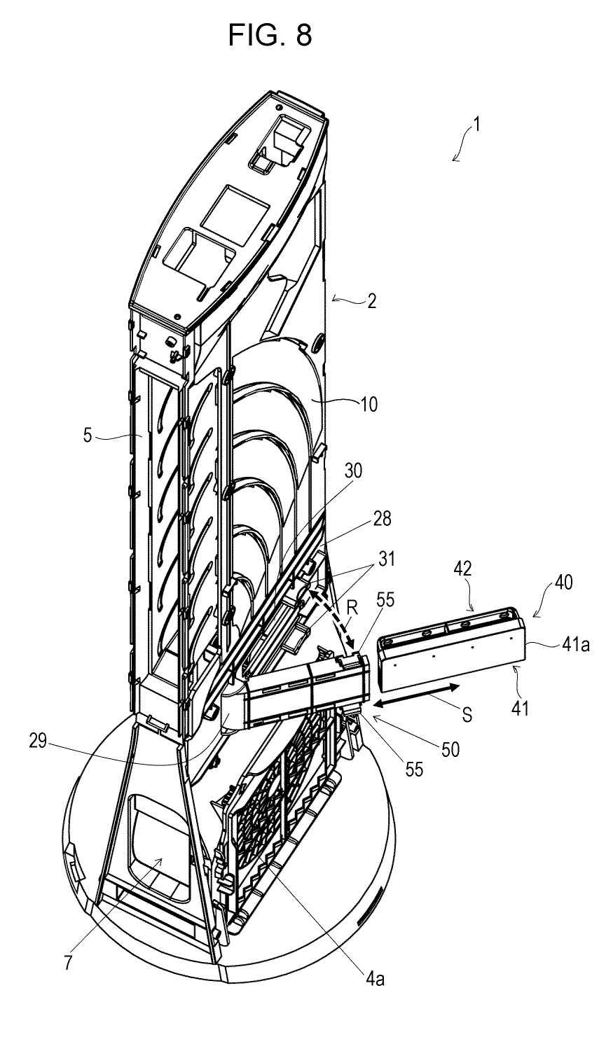

The inlet port 4 is formed of many small air holes 4b. On an inner surface side of the lower exterior cover 9L, a filter 4a (refer to FIG. 8) is provided so as to face the inlet port 4. An outlet port 5 is open at the front of the upper part of the body housing 2. An operating portion 6 is provided on the upper surface of the body housing 2.

The mount portion 3 is formed in a circular shape in a planar view, and supports the body housing 2 standing on the upper surface thereof. A power supply cord 8 is led from the circumferential surface of the mount portion 3. When a displacement motor not depicted and provided in the lower part of the body housing 2 is driven, the body housing 2 rotates in a horizontal plane with respect to the mount portion 3 about a rotation axis line extending in a vertical direction and provided at the center in a radial direction. With this, the body housing 2 undergoes to-and-fro displacement in a predetermined angle range to perform a swivel movement.

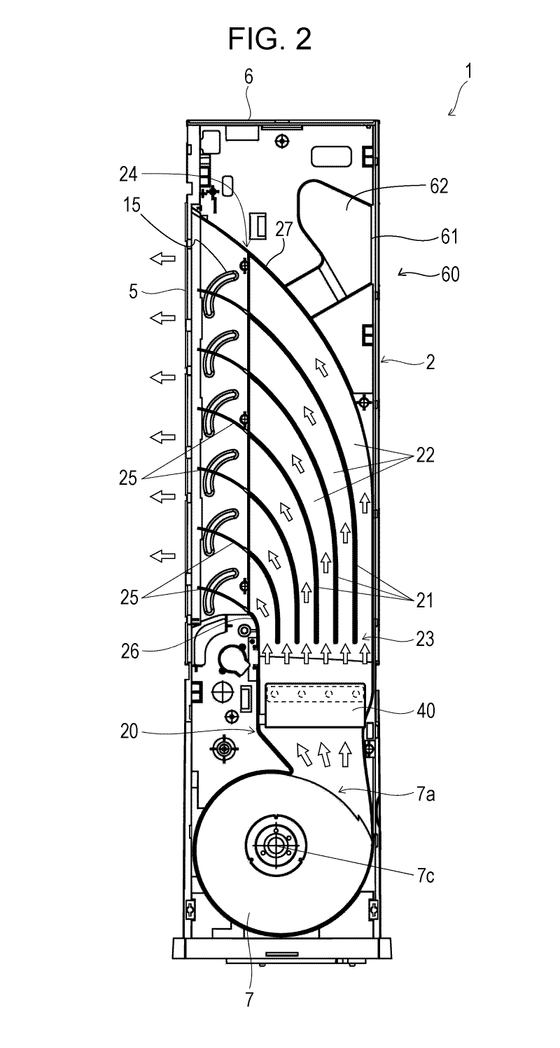

FIG. 2 is a side view depicting the inside of the ion transmitting device 1, depicting the state in which the exterior cover 9 and an inner cover 10 (refer to FIG. 13), which will be described further below, removed from the body housing 2. An air-blowing duct 20 extending in the vertical direction to make the inlet port 4 and the outlet port 5 communicate with each other is provided inside the body housing 2. An air-blowing fan 7 is disposed inside the air-blowing duct 20 and in the lower part of the body housing 2.

The air-blowing fan 7 is configured of a centrifugal fan such as a sirocco fan, and an intake port (not depicted) is disposed so as to face the inlet port 4. An exhaust port 7a of the air-blowing fan 7 is open upward. Air taken in from the inlet port 4 by driving the air-blowing fan 7 is distributed toward the outlet port 5. The air-blowing fan 7 may be configured of a turbofan, or may be configured of an axial-flow fan.

The air-blowing duct 20 has a plurality of divisional pathways 22 downstream from the air-blowing fan 7, extending upward, curved toward the outlet port 5 frontward, and obtained by division with partition plates 21. In the present embodiment, six divisional pathways 22 are provided, with five partition plates 21. The divisional pathways 22 are aligned in a line in a front-and-rear direction of the ion transmitting device 1 at an entrance portion 23, and are aligned in a line in a vertical direction at an exit portion 24.

The partition plates 21 form upper and lower wall surfaces for the respective divisional pathways 22, and six flexible airflow direction plates 25 are disposed at the outlet port 5 so as to extend a lower wall 26 for the partition plates 21 and the air-blowing duct 20. The airflow direction plates 25 each have an upstream end pivotally supported by the body housing 2, and each have a projecting portion (not depicted) projecting from the side surface fitting in a guide groove 15 provided in the body housing 2. With the projecting portion displaced in the guide groove 15, the orientation of the airflow direction plate 25 is changed.

Between the air-blowing fan 7 and the divisional pathways 22, an ion generator 40 which generates ions is disposed. Ions generated by the ion generator 40 are contained in airflows distributed through the air-blowing duct 20. Since the ion generator 40 is disposed downstream from the air-blowing fan 7, it is possible to prevent disappearance of ions due to collision with the air-blowing fan 7.

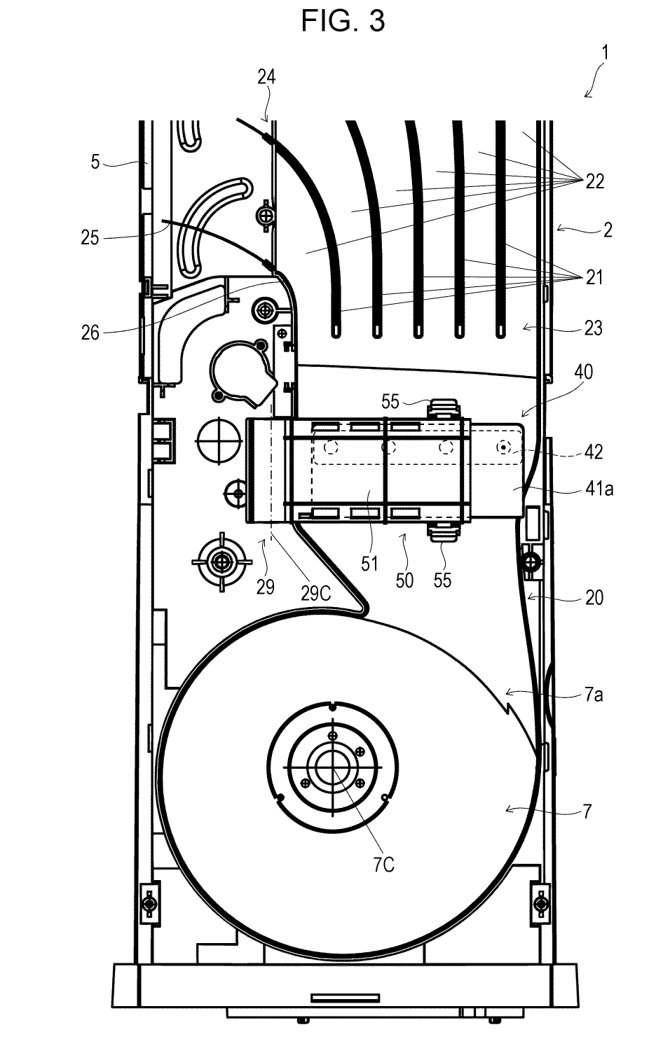

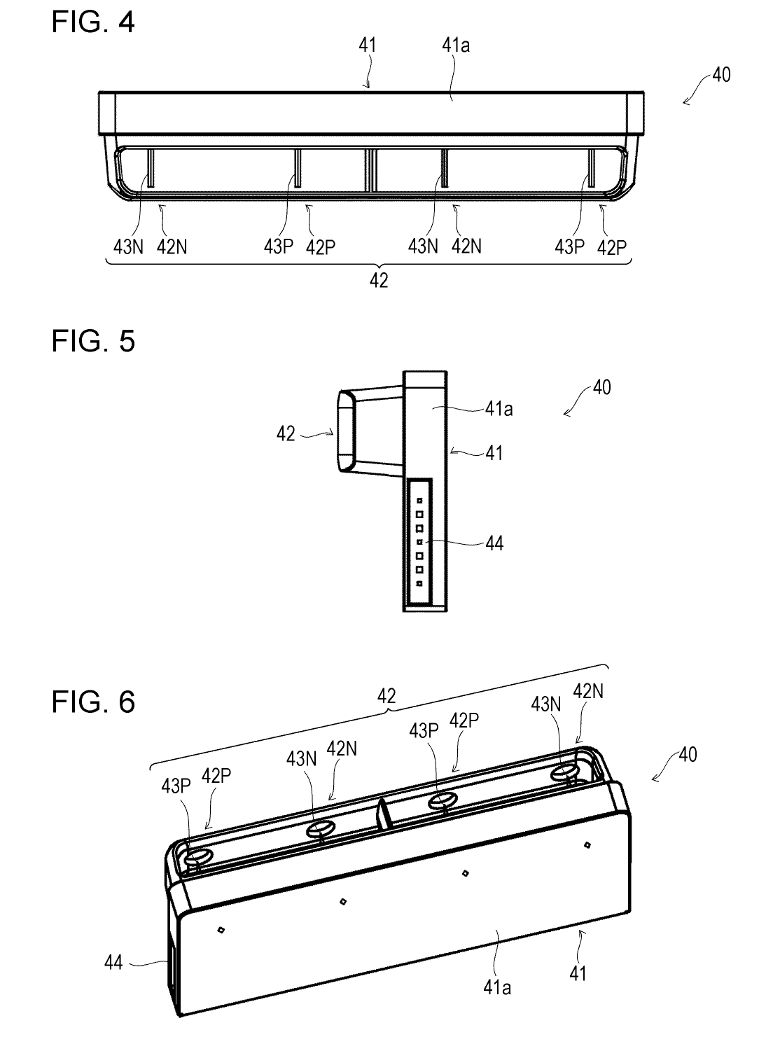

FIG. 3 is a partially-enlarged view of FIG. 2. Also, FIG. 4 to FIG. 6 are a top view, a side view, and a perspective view, respectively, of the ion generator 40. The ion generator 40 is held by a holder 50, and has a discharge electrode and other electronic components disposed inside a housing 41 and packaged. The holder 50 is mounted on a side plate 28 (refer to FIG. 8) covering the lower part of the air-blowing duct 20.

The ion generator 40 has a terminal portion 44 for electric power supply on a side surface of the housing 41, and includes a circuit portion (not depicted) and positive ion generating portions 42P and negative ion generating portions 42N. The positive ion generating portions 42P and the negative ion generating portions 42N of the ion generator 40 held by the holder 50 are disposed so as to face the air-blowing duct 20. Also, the circuit portion includes a high-voltage electricity generating circuit which generates a high-voltage electrical pulse when supplied with electric power from an external source.

The positive ion generating portions 42P each include a positive discharge electrode 43P, and the negative ion generating portions 42N each include a negative discharge electrode 43N. In the following description, the positive ion generating portions 42P and the negative ion generating portions 42N may be collectively referred to as an ion generating portion 42. The positive ion generating portions 42P and negative ion generating portions 42N project from a body portion 41a of the housing 41 toward outside (an air-blowing duct 20 side).

Two pairs of the positive discharge electrode 43P and the negative discharge electrode 43N are formed each in a needle shape and are disposed so as to be aligned with a predetermined space apart from each other and face the air-blowing duct 20 via an opening portion. Also, two pairs of the positive discharge electrode 43P and the negative discharge electrode 43N are aligned in a direction crossing an air distributing direction inside the air-blowing duct 20 to form a substantially right angle.

The positive ion generating portions 42P and the negative ion generating portions 42N are formed in the same structure. A high voltage generated by the high-voltage electricity generating circuit is supplied to each of the positive discharge electrode 43P and the negative discharge electrode 43N to cause discharge and transmit ions.

A voltage formed of an alternating-current waveform or an impulse waveform is applied to the positive discharge electrodes 43P and the negative discharge electrodes 43N. A positive voltage is applied to the positive discharge electrodes 43P, and hydrogen ions generated by corona discharge are bonded to moisture in the air to generate positive ions mainly formed of H⁺ (H₂O)m.

A negative voltage is applied to the negative discharge electrodes 43N, and oxygen ions generated by corona discharge are bonded to moisture in the air to generate negative ions mainly formed of O₂⁻ (H₂O)n. Here, m and n are any natural numbers. H⁺(H₂O)m and O₂⁻(H₂O)n are agglomerated on the surfaces of floating fungi and odor components in the air to take in these fungi and components.

And, as indicated by Formulas (1) to (3), [.OH] (hydroxyl radical) and H₂O₂ (hydrogen peroxide), which are active species, are agglomerated and generated by collision on the surfaces of microbes and so on to destroy floating fungi and odor components. Here, m′ and n′ are any natural numbers. Therefore, the ion generator 40 transmits positive ions and negative ions to the inside of the air-blowing duct 20, and the positive ions and the negative ions are transmitted from the outlet port 5. With this, for example, indoor sterilization and deodorization can be performed.

H⁺(H₂O)m+O₂⁻(H₂O)n→.OH+½O₂+(m+n)H₂O (1)

H⁺(H₂O)m+H⁺(H₂O)m′+O₂⁻(H₂O)n+O₂⁻(H₂O)n′→2.OH+O₂+(m+m′+n+n′)H₂O (2)

H+(H₂O)m+H⁺(H₂O)m′+O₂⁻(H₂O)n+O₂⁻(H₂O)n′→H₂O₂+O₂+(m+m′+n+n′)H₂O (3)

While the positive ions and negative ions are generated by the ion generator 40 in the present embodiment, only positive ions or negative ions may be generated.

Also, the ion generator 40 may be an electrostatic atomizer which generates ions formed of charged fine particle water. By the electrostatic atomizer, charged fine particle water containing radical components is generated. That is, a discharge electrode provided in the electrostatic atomizer is cooled by a Peltier device to generate dew condensation water on the surface of the discharge electrode. Next, when a minus high voltage is applied to the discharge electrode, charged fine particle water is generated from the dew condensation water. Also, from the discharge electrode, negative ions transmitted into the air together with the charged fine particle water are also generated.

Furthermore, the ion generator 40 may be an electrostatic atomizer which generates either one of positive ions or negative ions and fine particle water charged with a polarity opposite to the polarity of the positive ions or the negative ions. When negative ions or negatively-charged fine particle water is generated, a relaxation effect is said to be also produced, in addition to indoor sterilization and deodorization.

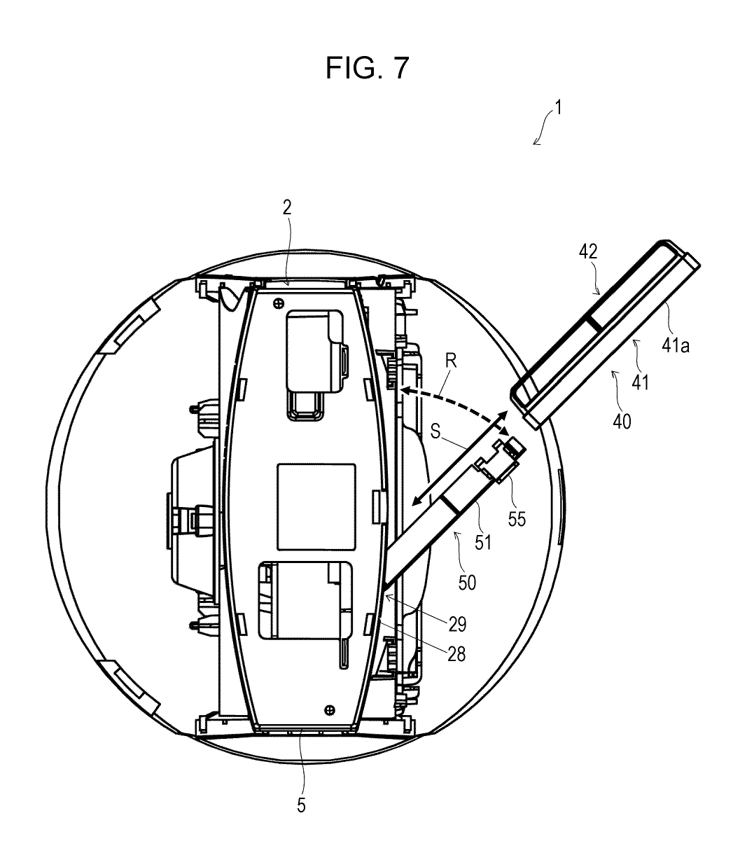

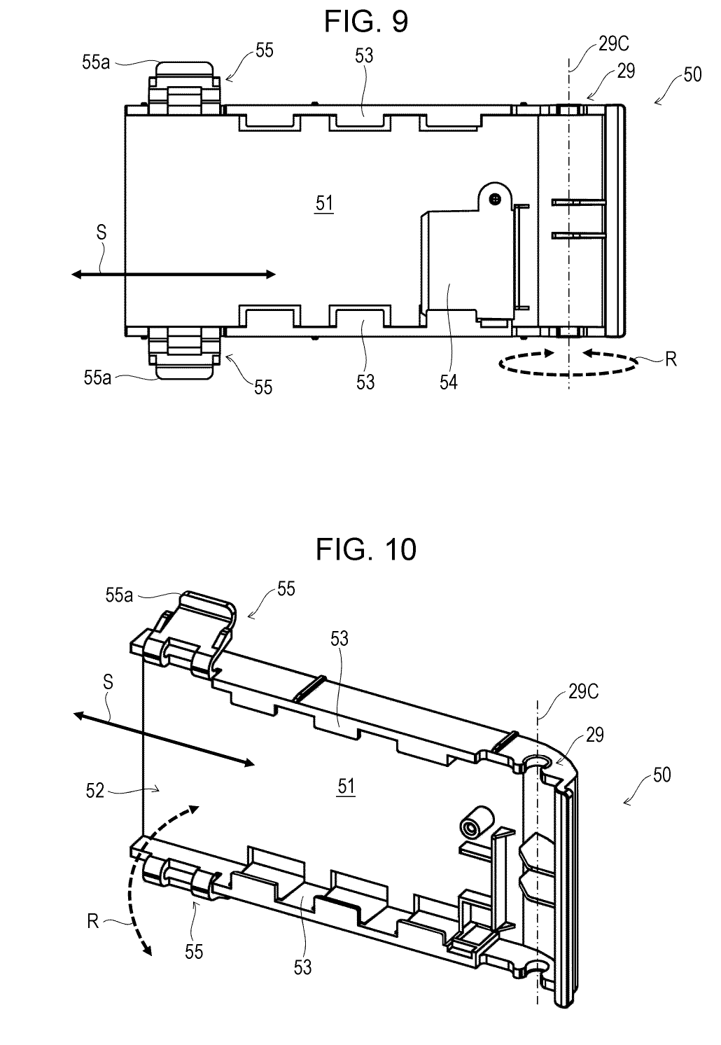

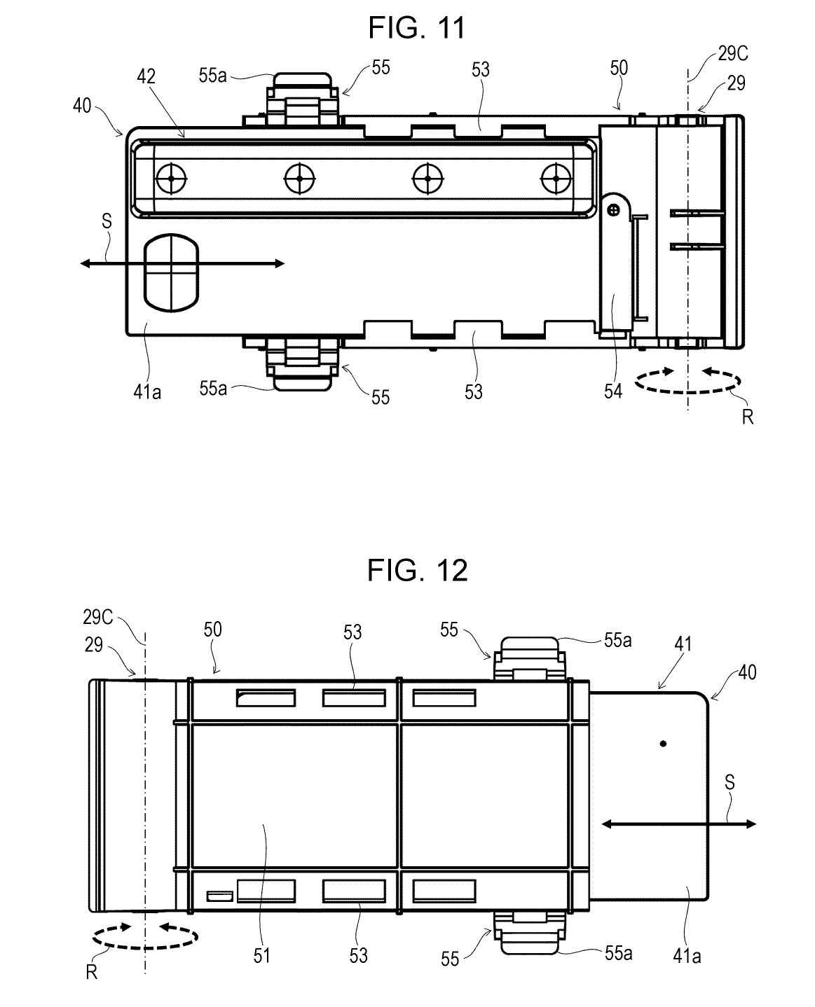

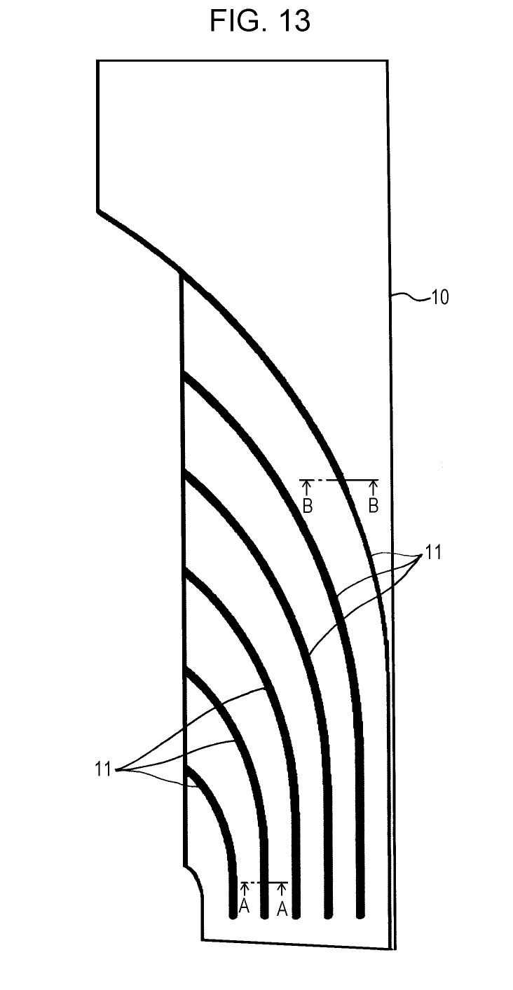

Next, details of the mount structure of the ion generator 40 are described by using FIG. 7 to FIG. 12 in addition to FIG. 3. FIG. 7 is a top view of the ion transmitting device 1, and FIG. 8 is a perspective view of the ion transmitting device 1 with the exterior cover 9 removed therefrom. FIG. 9 and FIG. 10 are a front view and a perspective view, respectively, of the holder 50. FIG. 11 and FIG. 12 are a front view and a rear view, respectively, of the ion generator 40 and the holder 50.

Note that, for the ion generator 40 and the holder 50, an air-blowing duct 20 side depicted in FIG. 9 and FIG. 11 is taken as front and an exterior cover 2 side depicted in FIG. 12 is taken as rear. Also, a broken arrow line R of FIG. 7 to FIG. 12 indicates a rotating direction of the holder 50, and a solid arrow line S indicates a sliding direction of the ion generator 40 with respect to the holder 50.

The holder 50 holds the ion generator 40 in the manner as described above, and is mounted on the side plate 28. The holder 50 and the ion generator 40 are provided on the side plate 28 on the right when the ion transmitting device 1 is viewed from a front surface side with the outlet port 5 open, as depicted in FIG. 3, FIG. 7, and FIG. 8. Note that the holder 50 may be mounted on the side plate 28 via another member provided to the side plate 28.

The holder 50 is provided with a hinge portion 29, which is a holder coupling portion rotatably coupled to the side plate 28 on the front surface side of the ion transmitting device 1. The hinge portion 29 is disposed ahead of the air-blowing duct 20. The exhaust port 7a of the air-blowing fan 7 formed of a sirocco fan is disposed so as to be decentered in a decentering direction behind a rotation axis line 7C. With this, the air-blowing duct 20 is led from the air-blowing fan 7 to have an imbalance to the rear part of the body housing 2. Thus, the hinge portion 29 is disposed in a vacant space ahead and opposite in direction to the decentering direction of the air-blowing duct 20, and cushioning of the hinge portion 29 with respect to the airflow distributed through the air-blowing duct 20 can be prevented.

The hinge portion 29 has a center axis line 29C in a vertical direction and extending in an airflow distributing direction of the air-blowing duct 20. With this, as indicated by the dotted arrow line R in FIG. 7, FIG. 8, and FIG. 10, the holder 50 can rotate in a substantially horizontal plate, with a rear end taken as a free end. Note that FIG. 7 and FIG. 8 depict the state in which the holder 50 is rotated so that the free end on a rear surface side of the holder 50 is separated away from the side plate 28.

The side plate 28 is open at a widow portion 30, and the ion generator 40 held by the holder 50 approaches and is separated away from the side plate 28 with rotation. The ion generating portion 42 of the ion generator 40 when approaching the side plate 28 is disposed so as to face the air-blowing duct 20 via the window portion 30. Here, the body portion 41a of the ion generator 40 is disposed outside the side plate 28.

That is, the holder 50 can rotate so as to take a position where the ion generating portion 42 faces the inside of the air-blowing duct 20 via the window portion 30 and a position where the ion generating portion 42 is separated away outward from the window portion 30. Here, the ion generating portion 42 may be disposed completely inside of the inner surface of the side plate 28 via the window portion 30 to face the inside of the air-blowing duct 20. Alternatively, a surface of the body portion 41a of the ion generator 40 facing the window portion 30 may approach the outer surface of the side plate 28 to cause the ion generating portion 42 to face the inside of the air-blowing duct 20. Alternatively, the tip of the ion generating portion 42 may approach the outer surface of the side plate 28 to cause the ion generating portion 42 to face the inside of the air-blowing duct 20.

The holder 50 includes an accommodating portion 52 for accommodating the ion generator 40 inside a rear surface wall 51. Also, the holder 50 includes a guide portion 53 on each of upper and lower parts of the accommodating portion 52, the guide portion 53 extending between a free end side and a hinge portion 29 side. As indicated by the solid arrow line S in FIG. 7 to FIG. 12, the ion generator 40 makes a sliding movement in a direction crossing the center axis line 29C along the guide portions 53 to form a substantially right angle.

With this, the ion generator 40 can be caused to make a sliding movement in the direction from the free end side toward the hinge portion 29 side of the holder 50 to be inserted into the accommodating portion 52. Also, the ion generator 40 can be removed from the accommodating portion 52 by being caused to make a sliding movement along the guide portions 53 in a direction opposite to the above-described direction.

The holder 50 includes a connector portion 54 on a hinge portion 29 side of the accommodating portion 52. The connector portion 54 is electrically connected to a control board or the like of the body housing 2, thereby receiving electric power and a control signal. When the ion generator 40 is inserted into the accommodating portion 52, the terminal portion 44 (refer to FIG. 5 and FIG. 6) of the ion generator 40 is also coupled to the connector portion 54 (refer to FIG. 11). With this, the ion generator 40 receives supply of electric power from the body housing 2 to receive a control signal. Note that depiction of the connector portion 54 is omitted in FIG. 10.

The holder 50 includes an engaging piece 55 on each of upper and lower parts on the free end side. Also, the side plate 28 includes engaging portions 31 at positions corresponding to the respective engaging pieces 55 at two positions (refer to FIG. 8). By pressing a knob portion 55a at the tip, each engaging piece 55 is elastically deformed to be engaged with or disengaged from the engaging portion 31.

When the engaging pieces 55 of the holder 50 having the ion generator 40 inserted therein are engaged with the engaging portions 31 of the side plate 28, the ion generating portion 42 is held as facing the inside of the air-blowing duct 20 via the window portion 30. With this, ions are transmitted from the ion generating portion 42 to the air-blowing duct 20 to cause ion transmitting operation by the ion transmitting device 1 to be performed.

FIG. 13 is a front view of the inner cover 10 covering the divisional pathways 22. The inner cover 10 is formed of a resin-molded article, and is removably disposed. With the inner cover 10, the side wall of the divisional pathways 22 is formed, and the flow-path area of the exit portion 24 is gradually narrowed with respect to the entrance portion 23 of the divisional pathways 22. With this, the air velocity of the airflow transmitted from the outlet port 5 can be increased to let the airflow spread indoors.

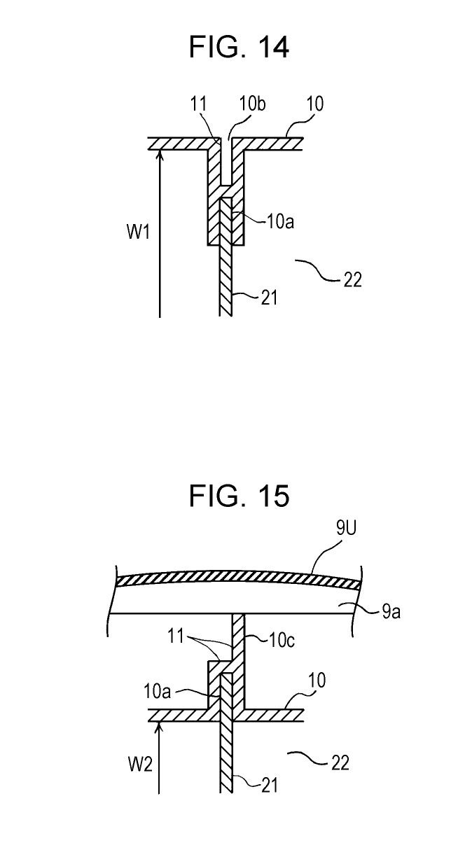

On the outer surface of the inner cover 10, marking portions 11 visually recognized at the time of mounting are formed along the partition plates 21. FIG. 14 and FIG. 15 are an A-A sectional view of FIG. 3 and a B-B sectional view thereof, respectively. FIG. 14 depicts a part near the entrance portion 23 (refer to FIG. 2) on an upstream side of the divisional pathways 22, and FIG. 15 depicts a part on a downstream side of the entrance portion 23.

The inner surface of the inner cover 10 determines a flow-path width of each divisional pathway 22, and a flow-path width W2 of the divisional pathway 22 on the downstream side in a lateral direction is formed smaller than a flow-path width W1 thereof on the upstream side. With this, the downstream flow-path area is narrowed with respect to the upstream of the divisional pathways 22.

In the inner surface of the inner cover 10, a fitting groove 10a that fits in the partition plate 21 is formed. On an upstream part with the larger flow-path width W1, both side walls of the fitting groove 10a are extended, and a groove portion 10b is provided in a recessed shape in the outer surface of the inner cover 10. The groove portion 10b forms the marking portion 11 along the partition plate 21.

On a downstream part with the smaller flow-path width W2, the rear surface of the fitting groove 10a projects from the outer surface of the inner cover 10. The rear surface of the fitting groove 10a forms the marking portion 11 along the partition plate 21. Also, in the downstream part, a rib 10c obtained by extending one side wall of the fitting groove 10a projects from the rear surface of the fitting groove 10a. With the rib 10c, clear visual recognition of the marking portion 11 can be made.

Since the upper exterior cover 9U is formed in a curved surface projecting outward, a reinforcing rib 9a for reinforcement is provided to the inner surface so as to extend in a direction crossing the partition plate 21. With the upper exterior cover 9U inserted, the reinforcing rib 9a abuts on the rib 10c of the inner cover 10. With this, deformation of the upper exterior cover 9U can be prevented when the upper exterior cover 9U is pressurized from outside. Note that the reinforcing rib 9a may abut on the rear surface of the fitting groove 10a by omitting the rib 10c.

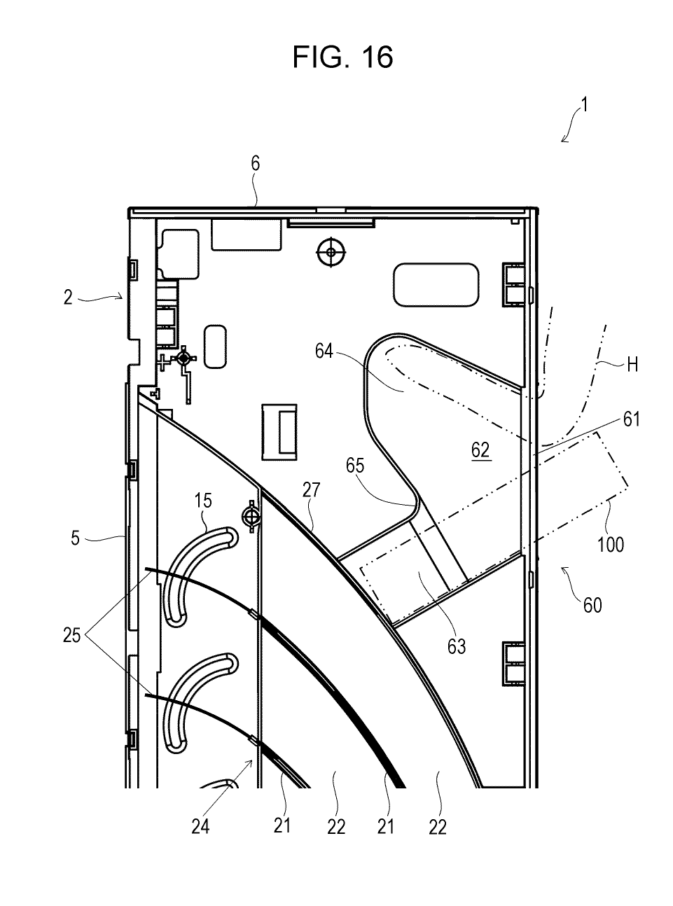

FIG. 16 is a side view depicting details of the upper part of FIG. 2. The ion transmitting device 1 is remotely operated by a remote control 100, and a remote control holding structure 60 is provided on an upper rear surface of the body housing 2 to accommodate and hold the remote control 100.

The remote control holding structure 60 includes a single insertion port 61 which is open in the upper rear surface of the body housing 2 and a holding hole 62 extending from the insertion port 61 toward the inside of the ion transmitting device 1. The insertion port 61 is formed of a rectangular opening of a size allowing the remote control 100 and a human finger H to be simultaneously inserted therein. The holding hole 62 is formed of a closed-end hole of a size allowing the remote control 100 and the human finger H to be simultaneously accommodated therein.

The holding hole 62 includes a remote control holding hole 63 (a remote control holding portion) and a hand-hook hole 64 (a hand-hook portion). The remote control holding hole 63 holds the remote control 100 inserted from the insertion port 61. In the hand-hook hole 64, the human finger H is hooked at the time of carrying the ion transmitting device 1. The remote control holding hole 63 is provided below the holding hole 62, and extends from the insertion port 61 in a diagonally downward direction. The hand-hook hole 64 is provided above the holding hole 62, and extends from the insertion port 61 in a diagonally upward direction.

A wall portion that is present at the deepest in the remote control holding hole 63 is formed as a partition wall that is common to the wall plate 27 on the most rear surface side of the air-blowing duct 20.

Inside the holding hole 62, a projecting portion 65 projecting toward an insertion port 61 side is provided. The projecting portion 65 is provided between the remote control holding hole 63 and the hand-hook hole 64, and sections the inside of the holding hole 62 into the remote control holding hole 63 and the hand-hook hole 64.

The holding hole 62 is formed to have a size allowing the remote control 100 accommodated in the remote control holding hole 65 to partially project outward from the insertion port 61. The remote control holding hole 65 may be formed to have a size allowing the entire remote control 100 to be accommodated inside the insertion port 61.

According to the present embodiment, the ion transmitting device 1 has the holder 50 which removably holds the ion generator 40 provided on the side plate 28 configuring one surface of the air-blowing duct 20. The holder 50 rotates so as to take a position where the ion generating portion 42 faces the inside of the air-blowing duct 20 via the window portion 30 and a position where the ion generating portion 42 is separated away outward from the window portion 30. With this, at the time of replacement of the ion generator 40, the holder 50 can be prevented from being removed from the body housing 2 of the ion transmitting device 1. Therefore, the ion generator 40 can be easily replaced, and a loss of the holder 50 can be prevented.

Also, since the ion transmitting device 1 includes the hinge portion 29, which is a holder coupling portion having the holder 50 mounted thereon and allowing rotation of the holder 50, rotation of the holder 50 with respect to the side plate 28 can be easily performed.

Furthermore, from the air-blowing fan 7 formed of a centrifugal fan to the rotation axis line 7C, the air-blowing duct 20 is led to have an imbalance in one (rearward) decentering direction. The hinge portion 29 of the holder 50 is disposed outside in a direction (forward) opposite to the decentering direction of the air-blowing duct 20. With this, the hinge portion 29 can be disposed in the vacant space inside the body housing 2 to reduce the size of the ion transmitting device 1. Still further, cushioning of the hinge portion 29 with respect to the airflow distributed through the air-blowing duct 20 can be prevented, and the air-blowing duct 20 can be formed so that air can be efficiently distributed.

Still further, since the holder 50 includes the guide portions 53 which cause the ion generator 40 to make a sliding movement at the time of attaching and removing the ion generator 40, the ion generator 40 can be easily attached to and removed from the holder 50.

Still further, since the guide portions 53 cause the ion generator 40 to make a sliding movement in a direction crossing the center axis line 29C of rotation of the holder 50 to form a substantially right angle, the ion generator 40 can be easily attached and removed even when the rotation range of the holder 50 is relatively narrow.

Still further, since the holder 50 includes the connector portion 54 which is electrically connected to the ion generator 40 together with the insertion of the ion generator 40 into the holder 50, the ion generator 40 and the connector 54 can be automatically coupled together, and these electrical connections can be easily established.

Still further, since the side plate 28 includes the window portion 30 through which the ion generating portion 42 passes at the time of an approach of the ion generator 40 due to rotation of the holder 50, the ion generating portion 42 faces the air-blowing duct 20 via the window portion 30, and ions can be easily transmitted to the airflow distributed through the air-blowing duct 20.

Still further, the ion generator 40 includes two pairs of the positive ion generating portion 42P and the negative ion generating portion 42N, and these positive ion generating portions 42P and the negative ion generating portions 42N are aligned in a direction crossing an air distributing direction of the air-blowing duct 20 to form a substantially right angle. With this, ions can be efficiently transmitted to the airflow distributed through the air-blowing duct 20.

Still further, the air-blowing duct 20 having the divisional pathways 22 obtained by division with the partition plates 21 is provided inside the body housing 2 with its outer surface formed of the removable exterior cover 9. Still further, the removable inner cover 10 which covers the divisional pathways 22 has the marking portions 11 on the outer surface along the partition plates 21.

With this, the inside of the air-blowing duct 20 including the divisional pathways 22 can be cleaned by removing the exterior cover 9 and the inner cover 10. Therefore, a decrease in air-blowing efficiency of the ion transmitting device 1 can be prevented, and the transmitted airflow can be also kept clean. Still further, after cleaning of the air-blowing duct 20, the inner cover 10 can be easily mounted by superposing the marking portions 11 on the partition plates 21, and usability of the ion transmitting device 1 can be improved. Note that even in an air blower which is not provided with the ion generator 40 and transmits an airflow from the outlet port 5, similar effects can be obtained from a similar structure.

Still further, the inlet port 4 is provided in the lower part of the body housing 2 and the outlet port 5 is provided in the upper front surface of the body housing 2, and the partition plates 21 have a lower part extending in a vertical direction and an upper part curved toward the outlet port 5. With this, the marking portions 11 formed along the partition plates 21 are curved toward the front, and the direction of mounting the inner cover 10 can be easily identified.

Still further, since the rear surfaces of the fitting grooves 10a in which the partition plates 21 fit project from the outer surface of the inner cover 10 to form the marking portions 11, the marking portions 11 can be easily formed by molding processing of the inner cover 10.

Still further, since the marking portions 11 each have the rib 10c obtained by extending the side wall of the fitting groove 10a and projecting from the rear surface of the fitting groove 10a, clearer visual recognition of the marking portions 11 can be made.

Still further, the reinforcing rib 9a projecting from the inner surface of the upper exterior cover 9U formed on the curved surface projecting outward and extending in a direction crossing the partition plate 21 is provided, and the reinforcing rib 9a abuts on the rib 10c of the marking portion 11 at the time of mounting the upper exterior cover 9U. With this, deformation of the upper exterior cover 9U can be prevented when the upper exterior cover 9U is pressurized from outside.

Still further, the downstream flow-path area of the divisional pathways 22 is narrowed by the inner cover 10 with respect to the upstream, and the marking portion 11 facing the upstream part is formed by the groove portion 10b provided by extending both side walls of the fitting groove 10a and being recessed in the outer surface of the inner cover 10. With this, the marking portions 11 can be easily formed, and the air velocity of the airflow transmitted from the outlet port 5 can be increased, and therefore the airflow is spread indoors.

Still further, since the ion generator 40 which transmits ions toward the air-blowing duct 20 on the downstream side of the air-blowing fan 7 is provided, indoor sterilization and so on can be performed by transmitting ions from the outlet port 5. Here, with the divisional pathways 22, ions can be uniformly transmitted from the outlet port 5. Still further, since the airflows distributed through the divisional pathways 22 can be smoothly curved by the flexible airflow direction plates 25, a pressure loss can be reduced.

Still further, since the air-blowing fan 7 is formed of a centrifugal fan, the airflow is guided in a direction of the upper wall of the air-blowing duct 20 by a centrifugal force of the centrifugal fan. Here, the imbalance of the airflow is prevented by the divisional pathways 22, and the airflow can be uniformly transmitted from the outlet port 5.

While each marking portion 11 is formed by the rear surface of the fitting groove 10a, the rib 10c, and the groove portion 10b in the present embodiment, the marking portions 11 may be formed by printing on the outer surface of the inner cover 10.

Still further, the holding hole 62 has the remote control holding hole 63 (remote control holding portion) which holds the remote control 100 and the hand-hook hole 64 (hand-hook portion) in which the human finger H is hooked at the time of carrying the ion transmitting device 1. With this, the remote control holding hole 63 and the hand-hook hole 64 are integrally formed to reduce the number of components, and the occupying space of the remote control holding hole 63 and the hand-hook hole 64 can be decreased.

Still further, the projecting portion 65 is provided inside the holding hole 62 for sectioning into the remote control holding hole 63 and the hand-hook hole 64. With this, the remote control holding hole 63 can be formed to have a shape and size corresponding to the remote control 100, and the hand-hook hole 64 can be formed to have a shape and size corresponding to the human finger H. Therefore, a suitable clearance is provided between the remote control holding hole 63 and the remote control 100 and between the human finger H accommodated in the hand-hook hole 42 and the remote control 100, and usability of the ion transmitting device 1 can be improved.

Still further, since the holding hole 62 is formed to have a size so that the remote control 100 accommodated in the holding hole 62 partially project outward from the insertion port 61, the remote control 100 can be easily removed.

Still further, the air-blowing duct 20 extends upward from the inlet port 4 of the lower part of the body housing 2 to be curved forward and coupled to the outlet port 5 that is open at the upper front surface, and the remote control holding structure 60 is provided on the upper rear surface of the body housing 2. With this, the remote control holding structure 60 can be formed by effectively utilizing the vacant space on the upper rear surface side of the body housing 2, and the size of the ion transmitting device 1 can be decreased.

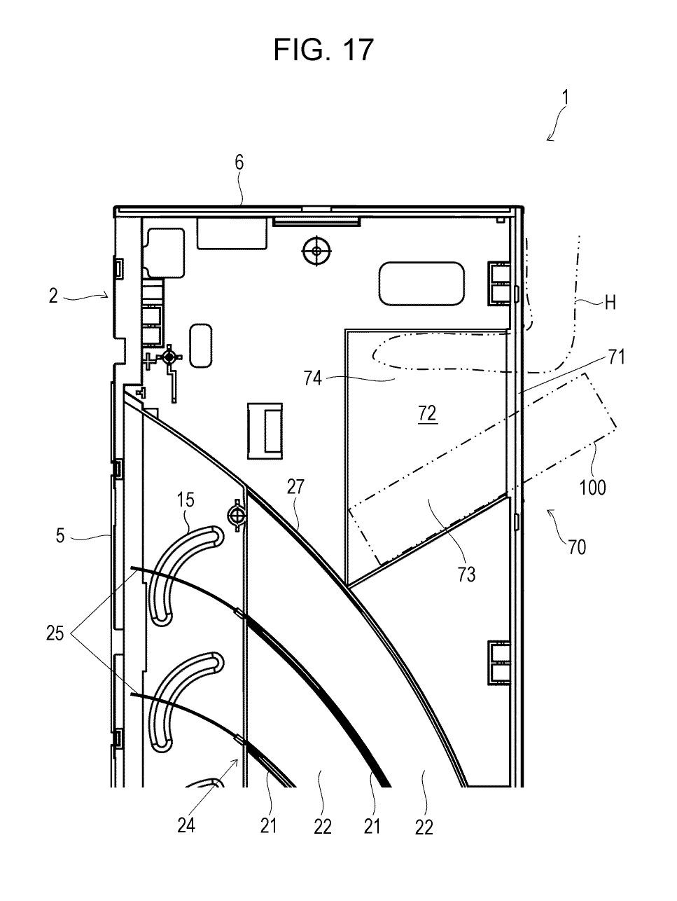

Next, FIG. 17 is a side view depicting the inside of an upper part of an ion transmitting device 1 of a second embodiment. For convenience of description, parts similar to those of the first embodiment depicted in FIG. 1 to FIG. 16 described above are provided with the same reference characters. In the present embodiment, a remote control holding structure 70 different in structure from the remote control holding structure 60 of the first embodiment is provided. Other portions are similar to those of the first embodiment.

The remote control holding structure 70 includes a single insertion port 71 that is open in the upper rear surface of the body housing 2 and a holding hole 72 extending from the insertion port 71 toward the inside of the ion transmitting device 1. The holding hole 72 is formed so that a vertical cross-section shape along a direction of inserting the remote control 100, that is, a cross-section shape viewed from sideward of the ion transmitting device 1, is a trapezoidal shape.

The holding hole 72 has a bottom surface formed of a tilted surface extending from a lower end of the insertion port 71 in a diagonally downward direction, and the remote control 100 inserted from the insertion port 71 is placed on the bottom surface. With this, a remote control holding portion 73 extending from the insertion port 71 in the diagonally downward direction to accommodate the remote control 100 is formed on a lower part of the holding hole 72.

The upper surface of the holding hole 72 is formed of a horizontal surface horizontally extending from an upper end of the insertion port 71, and the finger H inserted from the insertion port 71 is hooked. With this, a hand-hook portion 73 extending from the insertion port 71 in a lateral direction and in which the human finger H is hooked at the time of carrying the ion transmitting device 1 is formed in an upper part of the holding hole 72.

According to the present embodiment, the remote control holding portion 73 and the hand-hook portion 74 can be formed with a simple structure. Therefore, the size of the ion transmitting device 1 can be decreased, and cost can be decreased.

While the holder 50 and the ion generator 40 are provided on the right side when the ion transmitting device 1 is viewed from a front surface side in the first and second embodiments, the holder 50 and the ion generator 40 may be provided on a left side. Also, the holder coupling portion which rotatably supports the holder 50 may be structured of another coupling mechanism different from the hinge portion 29. Furthermore, while the hinge portion 29 is provided on the front part of the holder 50, the hinge portion 29 may be provided on a rear part, an upper part, or a lower part of the holder 50.

Still further, while the remote control holding structure 60, 70 is provided on the rear surface of the ion transmitting device 1, the remote control holding structure 60, 70 may be provided on a front surface, a left or right side surface, or an upper surface of the ion transmitting device 1.

Still further, while the remote control holding structure 60, 70 is provided to the ion transmitting device 1, the remote control holding structure 60, 70 may be provided to another portable electrical appliance including a remote control. Examples of the electrical appliance may be a television, an audio device, a fan, an electric heater, a humidifier, a dehumidifier, and so on. With these electrical appliances including the remote control holding structure 60, 70 similar to those described above, the number of components can be reduced, and occupying space can also be decreased.

### INDUSTRIAL APPLICABILITY

The present invention can be used in an air blower which transmits an airflow, an ion transmitting device which transmits an airflow containing ions, and an electrical appliance with a remote control.

### REFERENCE SIGNS LIST

ion transmitting device
2 body housing
3 mount portion
4 inlet port
5 outlet port
6 operating portion
7 air-blowing fan
7C rotation axis line
9 exterior cover
9a reinforcing rib
10 inner cover
10a fitting groove
10b groove portion
10c rib
11 marking portion
15 guide groove
20 air-blowing duct
21 partition plate
22 divisional pathway
25 airflow direction plate
28 side plate
29 hinge portion
29C center axis line
30 window portion
40 ion generator
42 ion generating portion
42P positive ion generating portion
42N negative ion generating portion
44 terminal portion
50 holder
52 accommodating portion
53 guide portion
54 connector portion
60, 70 remote control holding structure
61, 71 insertion port
62, 72 holding hole
63 remote control holding hole (remote control holding portion)
64 hand-hook hole (hand-hook portion)
65 projecting portion
73 remote control holding portion
74 hand-hook portion
100 remote control

1. An air blower comprising a body housing having an inlet port and an outlet port which are open; an air-blowing duct disposed inside the body housing, making the inlet port and the outlet port communicate with each other, and also having a plurality of divisional pathways obtained by division with partition plates; an air-blowing fan disposed upstream from the divisional pathways inside the air-blowing duct; a removable inner cover covering the divisional pathways and having an inner surface which forms a side wall of the air-blowing duct; and a removable exterior cover covering the inner cover to form an outer surface of the body housing;
wherein the inner cover has marking portions formed on an outer surface along the partition plates.

2. The air blower according to claim 1,
wherein the inlet port is provided in a lower part of the body housing and the outlet port is provided in a front surface of the body housing above the inlet port, and the partition plates have a lower part extending in a vertical direction and an upper part curved toward the outlet port.

3. The air blower according to claim 1 or 2,
wherein fitting grooves fitting in the partition plates are provided in the inner surface of the inner cover, and rear surfaces of the fitting grooves project from an outer surface of the inner cover to form the marking portions.

4. The air blower according to claim 3,
wherein the marking portions each have a rib obtained by extending a side wall of the fitting groove and projecting from the rear surface of the fitting groove.

5. The air blower according to claim 3,
wherein the exterior cover is formed on a curved surface projecting outward and has reinforcing ribs projecting from an inner surface and extending in a direction crossing the partition plates, and the reinforcing ribs abut on the marking portions when the exterior cover is mounted.

6. The air blower according to claim 3,
wherein a downstream flow-path area of the divisional pathways is narrowed by the inner cover with respect to an upstream, and the marking portion facing an upstream part of the divisional pathways has a groove portion provided by extending both side walls of the fitting groove and formed on the outer surface of the inner cover.

7. A remote control holding structure which accommodates and holds a remote control for performing a remote operation, the structure comprising a single insertion port which is open in an exterior of a body housing and allows the remote control and a human finger to be simultaneously inserted therein and a holding hole extending from the insertion port toward inside of the body housing,
wherein the holding hole has a remote control holding portion in a lower part and a hand-hook portion in an upper part, the remote control holding portion extending from the insertion port in a diagonally downward direction to hold the remote control and the hand-hook portion extending from the insertion port in a lateral direction or a diagonally upward direction and in which the human finger is hooked when the body housing is carried.

8. The remote control holding structure according to claim 7,
wherein a projecting portion projecting toward the insertion port side is provided inside of the holding hole, and sectioning into the remote control holding portion and the hand-hook portion is made by the projecting portion.

9. The remote control holding structure according to claim 7,
wherein the holding hole is formed so that a vertical cross-section shape along a direction of inserting the remote control is a trapezoidal shape.

10. The remote control holding structure according to claim 7,
wherein the holding hole is formed to have a size allowing the remote control accommodated in the holding hole to partially project outward from the insertion port.

11. An ion transmitting device comprising a body housing having an inlet port and outlet port which are open; an air-blowing duct making the inlet port and the outlet port communicate with each other; an air-blowing fan which distributes an airflow into the air-blowing duct; an ion generator having an ion generating portion which transmits ions to the air flow distributed through the air-blowing duct, a window portion formed of an opening provided on a side plate forming one surface of the air-blowing duct; and a holder which removably holds the ion generator;
wherein the holder can rotate so as to take a position where the ion generating portion faces inside of the air-blowing duct via the window portion and a position where the ion generating portion is separated away from the window portion.

12. The ion transmitting device according to claim 11,
wherein the air-blowing fan is formed of a centrifugal fan, the air-blowing duct is led from the air-blowing fan to have an imbalance in one decentering direction with respect to a rotation axis line of the air-blowing fan, and a holder coupling portion which rotatably supports the holder is disposed outside a direction opposite to the decentering direction with respect to the air-blowing duct.

13. The ion transmitting device according to claim 11,
wherein the holder includes a guide portion which causes the ion generator to make a sliding movement when the ion generator is attached or removed.

14. The ion transmitting device according to claim 13,
wherein the guide portion causes the ion generator to make a sliding movement in a direction crossing a center axis line of rotation of the holder.

15. The ion transmitting device according to claim 11,
wherein the holder includes a connector portion which is electrically connected to the ion generator together with insertion of the ion generator into the holder.

16. The ion transmitting device according to claim 11,
wherein the ion generator includes a plurality of said ion generating portions, and the plurality of said ion generating portions are aligned in a direction crossing an airflow distributing direction of the air-blowing duct.

17. An ion transmitting device comprising the air blower according to claim 1,
wherein an ion generator is provided which transmits ions toward the air-blowing duct on a downstream side of the air-blowing fan.

18. An ion transmitting device comprising the remote control holding structure according to claim 7 and an ion generator which generates ions,
wherein the ion transmitting device transmits an airflow containing ions.

19. The ion transmitting device according to claim 18,
wherein an air-blowing duct is provided which makes an inlet port which is open in a lower part of the body housing and an outlet port which is open in an upper front surface communicate with each other, the air-blowing duct extends upward from the inlet port and is curved forward to be coupled to the outlet port, and the remote control holding structure is provided to an upper rear surface of the body housing.

20. An electrical appliance comprising the remote control holding structure according to claim 7.
